(19) United States
(12) Patent Application Publication
(10) Pub. No.: US 2026/0166194 A1
(43) Pub. Date: Jun. 18, 2026

(54) Breathing Resistance Device having Aromatherapy Wick

(71) Applicant: Hunan Qinshi Industry and Trade Co., Ltd, Changsha (CN)

(72) Inventor: Wei Hu, Xianning (CN)

(21) Appl. No.: 19/010,208

(22) Filed: Jan. 6, 2025

(30) Foreign Application Priority Data
Dec. 14, 2024 (CN) 202411851031.8

(51) Int. Cl. A61L 9/12; A61M 15/00

(52) U.S. Cl. CPC A61L 9/127; A61M 15/0021

(57) ABSTRACT

The present disclosure discloses a breathing resistance device having an aromatherapy wick, comprising a tubular portion, an end of the tubular portion is provided with a connecting ring, an outer wall of the connecting ring is provided with a hanging chain, the other end of the tubular portion is connected with a dumbbell-shaped suction nozzle, the interior of the tubular portion is provided with an aromatherapy wick, an end of the dumbbell-shaped suction nozzle is provided with a connecting assembly, and the connecting assembly is used for connecting the dumbbell-shaped suction nozzle with the tubular portion.

### CLAIMS OF PRIORITY

This patent application claims priority from:
(1) CN invention patent application number is 202411851031.8 with a filing date of Dec. 14, 2024 and DAS code of ABC6.

### FIELD

The present disclosure relates to the technical field of breathing resistance device, particularly a breathing resistance device having an aromatherapy wick.

### BACKGROUND

A breathing resistance device is a multifunctional accessory that is both practical and decorative, and is usually designed in the shape of a small whistle that can be used for everyday carrying. With the addition of an aromatherapy wick function, the breathing resistance device can also diffuse a pleasant aroma and enhance the user's experience. The addition of the aromatherapy wick makes the breathing resistance device a functional and comfortable accessory for busy modern life. The design often includes a replaceable aromatherapy wick to ensure a long-lasting and even release of aroma to meet individual preferences and mood regulation needs. As a result, aromatherapy brings an atmosphere of relaxation and freshness that perfectly blends practical and aesthetic needs.

Conventional breathing resistance devices with aromatherapy wicks are very simple and convenient to use. First, the user can place an aromatherapy wick in a dedicated slot of a breathing resistance device to ensure that the aromatherapy wick fits snugly and can effectively release aroma. Then, with a hanging ring of the breathing resistance device, it can be attached to a key chain, backpack or necklace, and carried around. The airflow drives the aroma to diffuse through a built-in air channel, providing a long-lasting aromatic experience. The replacement of the aromatherapy wick is also very simple, the user only needs to remove the old piece and put in a new one, and the user can continue to enjoy fresh aroma and bring a soothing aromatic effect in daily use.

Conventional breathing resistance devices are designed with a sealing structure to ensure the long-lasting release of aroma. The aromatherapy wicks are usually fixed inside the breathing resistance devices or maintained by a press-fit design to prevent the aroma from leaking out or loosening the aromatherapy wicks, making the replacement process of the aromatherapy wicks cumbersome and inconvenient.

### SUMMARY

In order to remedy the above deficiencies, the present disclosure provides a breathing resistance device having an aromatherapy wick, which is intended to improve the problem that conventional breathing resistance devices make the replacement process of the aromatherapy wicks cumbersome and inconvenient due to their sealing characteristics.

In order to achieve the above purpose, the present disclosure provides the following technical solution: a breathing resistance device having an aromatherapy wick, comprising a tubular portion, an end of the tubular portion is provided with a connecting ring, an outer wall of the connecting ring is provided with a hanging chain, the other end of the tubular portion is connected with a dumbbell-shaped suction nozzle, the interior of the tubular portion is provided with an aromatherapy wick, an end of the dumbbell-shaped suction nozzle is provided with a connecting assembly, the connecting assembly is used for connecting the dumbbell-shaped suction nozzle with the tubular portion. The length of the dumbbell-shaped suction nozzle to the tubular portion is 40 to 80 mm, and the diameter thereof is 3 to 10 mm. The aromatherapy wick can be dripped with aromatic essential oil according to demand. The hanging chain conveniently fixes the tubular portion on the user. The length and diameter of the tubular portion and the configuration of the aromatherapy wick are used for restricting the user's exhalation, so that the user's breathing resistance increases to a desired breathing rate;

The connecting assembly comprises a connecting piece, the outer wall of the connecting piece is provided with an external thread, and the inner wall of the tubular portion is provided with an internal thread.

Furthermore, the interior of the tubular portion is provided with an air channel I, an end of the tubular portion is provided with a first air hole, an interior of the connecting piece is provided with an air channel II, an interior of the dumbbell shaped nozzle is provided with a dumbbell shaped channel, an end of the dumbbell shaped nozzle is provided with a second air hole, the outer wall of the connecting piece is provided with a sealing assembly, the sealing assembly is used for sealing between the connecting piece and the tubular portion, and the tubular portion has a round, oval, square, polygonal or irregular shape.

Furthermore, the sealing assembly comprises a sealing ring, an inner wall of the sealing ring is fixedly connected to the outer wall of the connecting piece, and an end of the tubular portion is provided with a limit slot.

Furthermore, the dumbbell-shaped channel, the air channel II and the air channel I are used for increasing resistance and slowing down the user's breathing rhythm.

Furthermore, the dumbbell-shaped channel, the air channel II and the air channel I can reduce the user's anxiety or stress and calm the user down.

Furthermore, the dumbbell-shaped channel can effectively regulate the airflow rate, so that the whistle can produce a more even and stable airflow output when used.

Furthermore, the dumbbell-shaped channel is designed to be thin in the middle and wide on both sides, so that the air flows more intensively at the thin and small channel in the middle, and after the airflow enters the wide part on both sides, the airflow rate gradually slows down, and the aroma is gradually diffused in this process.

Furthermore, the outer wall of the sealing ring is slidably connected to the inner wall of the limit slot, and the sealing ring is used for sealing between the connecting piece and the tubular portion.

The present disclosure has the following beneficial effects:
1. In the present disclosure, first, the tubular portion is conveniently carried through the connecting ring and the hanging chain, and then the internal aromatherapy wick is conveniently replaced through the external thread on the outer wall of the connecting piece and the internal thread on the inner wall of the tubular portion, and meanwhile, through the convenient use of the dumbbell-shaped suction nozzle, the problem of conventional breathing resistance devices making the replacement process of aromatherapy wicks cumbersome and inconvenient due to sealing characteristics thereof is solved, it achieves that the aromatherapy wick can be easily replaced, the operation of which is simple and convenient, and also ensures that the aroma is always fresh and even, and furthermore, it can help to regulate the mood, reduce stress and anxiety, and enhance the overall use experience through the aromatic air.
2. In the present disclosure, first, air is transported through the first air hole, the air channel I and the air channel II, and the air will be accompanied by part of the aroma after passing through the aromatherapy wick, and then with the wide parts of the front and rear sides of the dumbbell-shaped channel and the thin and small channel in the middle, the air and the aroma are fully mixed to achieve a stable release of aroma, preventing the aroma being too strong or too light, and the aroma in the whole space is evenly distributed to achieve a long-lasting and stable aromatic experience.

### BRIEF DESCRIPTION OF THE DRAWINGS

FIG. 1 is a schematic diagram of the three-dimensional structure of the breathing resistance device having an aromatherapy wick provided by the present disclosure:

FIG. 2 is a schematic diagram of the interior structure of the tubular portion of the breathing resistance device having an aromatherapy wick provided by the present disclosure;

FIG. 3 is a schematic diagram of the interior structure of the dumbbell-shaped suction nozzle of the breathing resistance device having an aromatherapy wick provided by the present disclosure.

### REFERENCE SIGNS

1. a tubular portion; 2. a connecting ring; 3. a hanging chain; 4. a dumbbell-shaped suction nozzle; 5. a first air hole; 6. an internal thread; 7. an air channel I; 8. a connecting piece; 9. an aromatherapy wick; 10. an external thread; 11. a second air hole; 12. a dumbbell-shaped channel; 13. a sealing ring; 14. a limit slot; 15. an air channel II.

### DETAILED DESCRIPTION

The technical solutions in the embodiments of the present disclosure will be clearly and completely described below in conjunction with the accompanying drawings in the embodiments of the present disclosure. Obviously, the described embodiments are only a portion of the embodiments of the present disclosure and not all of the embodiments. Based on the embodiments in the present disclosure, all other embodiments obtained by those skilled in the art without making creative labor fall within the protection scope of the present disclosure.

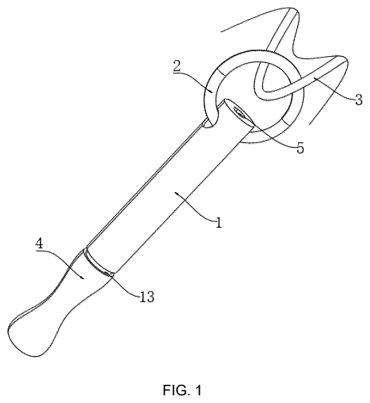

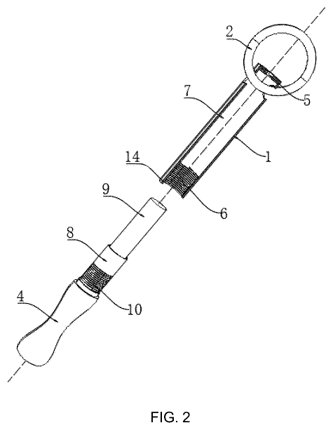

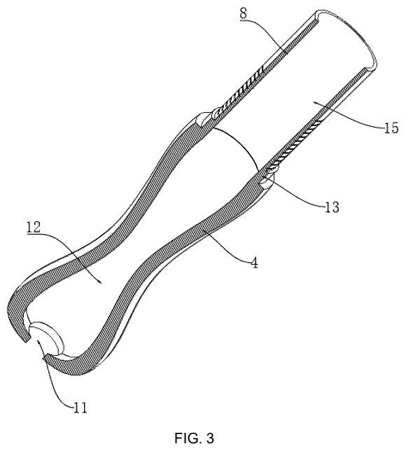

With reference to FIG. 1 to FIG. 3, the present disclosure provides one embodiment: a breathing resistance device having an aromatherapy wick, comprising a tubular portion 1, and the tubular portion 1 may be provided as a metal cylinder. An end of the tubular portion 1 is provide with a connecting ring 2, the outer wall of the connecting ring 2 is provided with a hanging chain 3, and it is convenient for the user to hang it on the body by the connecting ring 2 and the hanging chain 3, which is convenient to carry and use. The other end of the tubular portion 1 is provided with a dumbbell-shaped suction nozzle 4. The interior of the tubular portion 1 is provide with an aromatherapy wick 9. An end of the dumbbell-shaped suction nozzle 4 is provided with a connecting assembly for connecting the dumbbell-shaped suction nozzle 4 with the tubular portion 1. The length of the dumbbell-shaped suction nozzle 4 to the tubular portion is 40 to 80 mm, and the diameter thereof is 3 to 10 mm. Comfort is ensured by adapting the dumbbell-shaped suction nozzle 4 and the tubular portion 1 to the human body. The aromatherapy wick 9 can be dripped with aromatic essential oil according to demand to meet the aroma needs of different users and improve the wearer's comfort. The hanging chain 3 conveniently fixes the tubular portion 1 on the user. The length and diameter of the tubular portion 1 and the structure of the aromatherapy wick 9 are used for restricting the user's exhalation, so that the user's breathing resistance increases to a desired breathing rate. The connecting assembly comprises a connecting piece 8, the outer wall of the connecting piece 8 is provided with an external thread 10, and the inner wall of the tubular portion 1 is provided with an internal thread 6. Through the matching of threads, the connecting piece 8 can be firmly connected with the tubular portion 1, which is easy to disassemble and maintain, so that the air can flow more smoothly when passing through, and it can be convenient to replace the aromatherapy wick 9. The main body of the aromatherapy wick 9 is a porous material, and gas can pass through the first air hole 5, the air channel I 7, the aromatherapy wick 9 and the dumbbell-shaped channel 12 to reach the second air hole (11).

The inner diameter of the tubular portion 1 is larger than the inner diameter of the air channel II 15, an end of the aromatherapy wick 9 extends into the air channel II 15, and the air channel II 15 is used for fixing or clamping the aromatherapy wick 9.

With reference to FIG. 1 to FIG. 3, the interior of the tubular portion 1 is provided with an air channel I 7, an end of the tubular portion 1 is provided with a first air hole 5, through which the outside air is guided to enter the air channel I 7, and the diffusion of aroma is enhanced through the air flow. The interior of the connecting piece 8 is provided with an air channel 15. The interior of the dumbbell-shaped suction nozzle 4 is provided with a dumbbell-shaped channel 12, and the dumbbell-shaped channel 12 allows the aroma to diffuse. An end of the dumbbell-shaped suction nozzle 4 is provided with a second air hole 11, and the air continues to flow and output through the dumbbell-shaped suction nozzle 4 and the second air hole 11. The outer wall of the connecting piece 8 is provided with a sealing assembly, and the sealing assembly is used for sealing between the connecting piece 8 and the tubular portion 1. The tubular portion 1 has a round, oval, square, polygonal, or irregular shape. The sealing assembly comprises a sealing ring 13, the inner wall of the sealing ring 13 is fixedly connected to the outer wall of the connecting piece 8. The end of the tubular portion 1 is provided with a limit slot 14. The sealing assembly can ensure that the aroma does not leak and improve the efficiency of use. The dumbbell-shaped channel 12, the air channel II 15 and the air channel I 7 form a breathing channel that is used for increasing resistance and slowing down the user's breathing rhythm. The dumbbell-shaped channel 12, the air channel II 15 and the air channel I 7 can reduce the user's anxiety or stress and calm the user down. The dumbbell-shaped channel 12 can effectively regulate the airflow rate, so that the whistle can produce a more even and stable airflow output when used. The dumbbell-shaped channel 12 is designed to be thin in the middle and wide on both sides, that is, the cross-sectional area of the dumbbell-shaped channel 12 gradually decreases from an end away from the tubular portion 1 to the middle of the dumbbell-shaped passage 12, and then gradually increases from the middle of the dumbbell-shaped passage 12 to an end close to the tubular portion 1, so that the air flows more intensively at the thin and small channel in the middle, and after the airflow enters the wide part on both sides, the airflow rate gradually slows down, and the aroma is gradually diffused in this process. The outer wall of the sealing ring 13 is slidably connected to the inner wall of the limit slot 14, and the sealing ring 13 is used for sealing between the connecting piece 8 and the tubular portion 1.

Principle of operation: when a breathing resistance device with an aromatherapy wick needs to be used, firstly, the external thread 10 provided on the outer wall of the connecting piece 8 is matched with the internal thread 6 of the inner wall of the tubular portion 1 to realize the disassembly and assembly between the tubular portion 1 and the connecting piece 8, so that the aromatherapy wick 9 inside the connecting piece 8 and the tubular portion 1 is conveniently replaced. During the above process, through the installation of the connecting piece 8, the sealing ring 13 can be driven to move to the interior of the limit slot 14 preset in the tubular portion 1, so that the sealing ring 13 is used for sealing between the tubular portion 1 and the connecting piece 8. Then, when in use, the dumbbell-shaped suction nozzle 4 is put into the mouth to cooperate with the first air hole 5 for air intake, and cooperate with the air channel I 7 and air channel II 15 for air transportation, and during this process, aroma is added to the air through the aromatherapy wick 9. When the air with additional aroma is transported to the interior of the dumbbell-shaped channel 12, the airflow rate will gradually slow down when passing through the wide part of one side of the interior of the dumbbell-shaped channel 12, then the air will pass through the thin and small part in the middle of the dumbbell-shaped channel 12, and finally the air will be slowed down again through the wide part of the other side of the interior of the dumbbell-shaped channel 12, and in this process, the air and the aroma will be gradually diffused, and the effect of aromatherapy diffusion will be enhanced.

Finally, it should be noted that the above are only preferred embodiments of the present disclosure and are not used to limit the present disclosure. Although the present disclosure is described in detail with reference to the foregoing embodiments, those skilled in the art could still modify the technical solutions described in the foregoing embodiments, or replace some of the technical features therein. Any modification, equivalent replacement, improvement, etc., made within the spirit and principles of the present disclosure shall be included in the protection scope of the present disclosure.

1. A breathing resistance device having an aromatherapy wick, comprising a tubular portion (1), wherein: an end of the tubular portion (1) is connected with a dumbbell-shaped suction nozzle (4), an interior of the tubular portion (1) is provided with the aromatherapy wick (9), an end of the dumbbell-shaped suction nozzle (4) is provided with a connecting piece (8), and the connecting piece (8) is used for connecting the dumbbell-shaped suction nozzle (4) with the tubular portion (1), and the aromatherapy wick (9) has aromatic essential oil,
wherein an other end of the tubular portion (1) away from the dumbbell-shaped suction nozzle (4) is provided with a connecting ring (2), an outer wall of the connecting ring (2) is provided with a hanging chain (3), an outer wall of the connecting piece (8) is provided with an external thread (10), and an inner wall of the tubular portion (1) is provided with an internal thread (6),
wherein the interior of the tubular portion (1) is provided with an air channel I (7), an other end of the tubular portion (1) is provided with a first air hole (5), an interior of the connecting piece (8) is provided with an air channel II (15), an interior of the dumbbell shaped nozzle (4) is provided with a dumbbell shaped channel (12), an end of the dumbbell shaped nozzle (4) is provided with a second air hole (11), and an end of the aromatherapy wick (9) extends into the air channel II (15),
wherein the dumbbell-shaped channel (12) includes a first semi-channel and a second semi-channel, one end of the second semi-channel directly connected with the first semi-channel without an intermediate section of constant cross-sectional area, the cross-sectional area of the first semi-channel gradually decreases from one end away from the tubular portion (1) to another end connected with the second semi-channel, and the cross-sectional area of the second semi-channel gradually increases from the end connected with the first semi-channel to another end close to the tubular portion (1), the cross-sectional area of the air channel II (15) and the cross-sectional area of the air channel I (7) remain unchanged,
wherein the dumbbell-shaped channel (12), the air channel II (15) and the air channel I (7) form a breathing channel that is used for increasing resistance and slowing down a user's breathing rhythm,
wherein the direct connection between the first semi-channel and the second semi-channel causes airflow to accelerate continuously through the first semi-channel and decelerate continuously through the second semi-channel without stabilization, thereby enhancing aroma diffusion and breathing resistance control.

2-3. (canceled)

4. The breathing resistance device having an aromatherapy wick according to claim 1, wherein an outer wall of the connecting piece (8) is provided with a sealing assembly, the sealing assembly is used for sealing between the connecting piece (8) and the tubular portion (1), the sealing assembly comprises a sealing ring (13), an inner wall of the sealing ring (13) is fixedly connected to the outer wall of the connecting piece (8), and an other end of the tubular portion (1) is provided with a limit slot (14).

5. (canceled)

6. The breathing resistance device having an aromatherapy wick according to claim 1, wherein the aromatherapy wick (9) is a porous material, and gas can pass through the first air hole (5), the air channel I (7), the aromatherapy wick (9) and the dumbbell-shaped channel (12) to reach the second air hole (11).

7. (canceled)

8. The breathing resistance device having an aromatherapy wick according to claim 1, wherein an inner diameter of the tubular portion (1) is greater than an inner diameter of the air channel II (15), and the air channel II (15) is used for fixing the aromatherapy wick (9).

9. The breathing resistance device having an aromatherapy wick according to claim 4, wherein an outer wall of the sealing ring (13) is slidably connected to an inner wall of the limit slot (14), and the sealing ring (13) is used for sealing between the connecting piece (8) and the tubular portion (1).

10. The breathing resistance device having an aromatherapy wick according to claim 1, wherein a length of the dumbbell-shaped suction nozzle (4) to the tubular portion (1) is 40 to mm, and a diameter thereof is 3 to 10 mm.
